(19) United States
(12) Patent Application Publication
(10) Pub. No.: US 2009/0304784 A1
(43) Pub. Date: Dec. 10, 2009

(54) SEAMLESS CAPSULES CONTAINING HIGH AMOUNTS OF POLYUNSATURATED FATTY ACIDS AND A FLAVOURING COMPONENT

(75) Inventors: Jean Mane, Grasse (FR); Jean-Michel Hannetel, Grasse (FR); Pascal Dailland, Grasse (FR); Annaig Falchun, Bar Sur Loup (FR)

Correspondence Address: YOUNG & THOMPSON, 209 Madison Street, Suite 500, ALEXANDRIA, VA, 22314, US

(73) Assignee: V. MANE FILS, BAR SUR LOUP (FR)

(21) Appl. No.: 12/375,308

(22) PCT Filed: Jul. 25, 2007
(86) PCT No.: PCT/EP07/57672

§ 371 Date: Apr. 27, 2009

Related U.S. Application Data
(60) Provisional application No. 60/833,812, filed on Jul. 28, 2006.

(51) Int. Cl. A61K 31/20; A61K 9/48; A61J 3/07

(52) U.S. Cl. 424/451; 514/560; 264/4

(57) ABSTRACT

A seamless capsule includes a core and a shell, wherein the core includes at least one polyunsaturated fatty acid, and at least one flavouring component, the process for manufacturing the capsule and products containing the capsule are also disclosed.

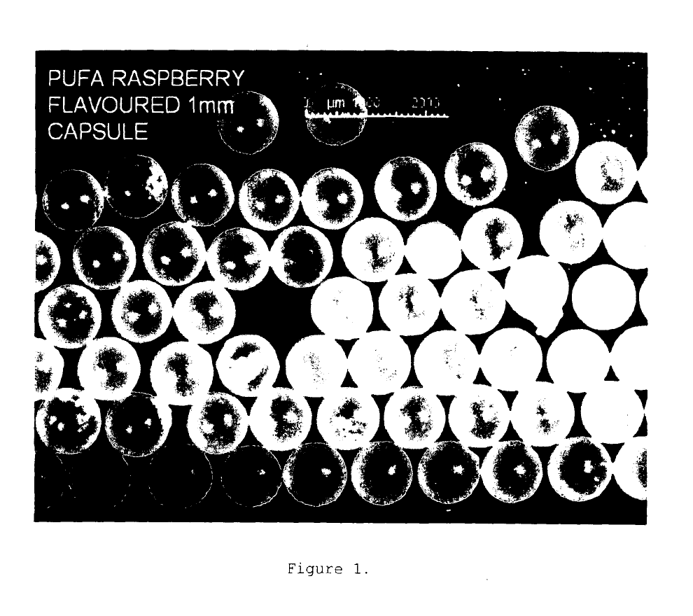

### FIELD

This invention relates to the field of animal, including human, nutrition and health, and is related to nutraceuticals, food and pharmaceutical products enriched in unsaturated fatty acids, preferably in polyunsaturated fatty acids (hereinafter referred to as “PUFAs”). More precisely, this invention relates to a delivery system of polyunsaturated fatty acids in the form of capsules, to a process for manufacturing said capsules, to a composition useful for the manufacturing of said capsules, containing fatty acids, preferably polyunsaturated fatty acids, and to food including pet food, dietetic, nutraceutic and pharmaceutical products including said delivery system of PUFAs.

### BACKGROUND

The beneficial effects of polyunsaturated fatty acids (PUFAs) on human health are known. Amongst the PUFAs, long-chain omega-3 fatty acids such as eicosapentanoic acid (EPA) or docosahexaenoic acid (DHA), for example, were shown to provide health benefits and are of interest in pharmaceutical products, nutritional products or supplements and in food products, especially in nutraceutic food products. Food fortification is more and more needed in food product and the main issue for such product is to supply a pleasant taste and an adequate shelf life and delivery for nutriments.

### PRIOR ART

However, PUFAs are very sensitive to oxidation and their degradation leads to the release of repellent fishy odours and tastes. WO2006/067647 seeks to avoid the degradation of PUFA by providing encapsulated PUFAs; in WO2006/067647, PUFAs are encapsulated into rods obtained from the extrusion through a die of a mixture of carbohydrate syrup emulsified with PUFAs. However, this process leads to very small rods having each low amounts of PUFAs encapsulated in each rods (around 15%).

There is still a need for providing PUFAs, under a non-oxidized form and under a form where the fishy odour and taste of PUFAs is either not perceptible or masked or where the odour of PUFAs is not perceptible and a pleasant aroma is released instead. One goal of this invention also is to provide a delivery system having high amounts of PUFA encapsulated therein, and including a flavouring component which make the product more pleasant for the consumer and avoid interaction of PUFA with other food ingredients.

### DETAILED DESCRIPTION

This invention thus relates to a delivery system of polyunsaturated fatty acids in the form of capsules comprising a liquid core and a solid shell, the core containing PUFAs and at least one flavouring agent, and the shell being a barrier to outer air and preventing the core of the shell from oxidation due to the air.

Thus, the invention relates to a seamless capsule comprising a core and a shell, wherein the core includes at least one PUFA and at least one flavouring component. According to a preferred embodiment, the core of the seamless capsule of the invention further includes at least one diluent wherein both the at least one PUFA and the at least one flavouring component may be solubilized.

In the meaning of this invention, by seamless capsule is meant a spherical or substantially spherical delivery system of at least one PUFA, said capsule comprising a liquid inner core and a solid outer spherical shell.

By polyunsaturated fatty acid (PUFA) is meant a fatty acid having at least two double bonds on the more-than-8-carbon aliphatic chain. PUFA are generally grouped into two series, on the basis of the position of the terminal double bond being 3C or 6C from the terminal carbon atom of the fatty acid chain and and often referred to as omega-3 and omega-6 fatty acid. Omega-3 fatty acid includes, but are not limited to linolenic acid (LA), alpha-linolenic acid (ALA), eicosapentaenoic acid (EPA) and docosahexaenoic acid (DHA) ; omega-6 fatty acid includes, but are not limited to linoleic acid (LA), gamma-linolenic acid (GLA), arachidonic acid (AA) and dihomo-gamma-linolénique (DGLA). In the meaning of the invention, the terms “fatty acid” or “polyunsaturated fatty acid” or “PUFA” also includes the salts, esters and glycerid, especially triglycerid, derivates of said acids.

According to an embodiment, the diameter of each seamless capsule of the invention is from 0.8 to 8 mm, preferably from 1 to 3 mm.

Advantageously, the dry weight of the capsule according to the invention is from 0.25 to 260 mg, preferably from 0.5 to 100 mg, and more preferably from 0.7 to 15 mg.

According to a preferred embodiment of the invention, the core represents 50 to 92%, preferably from 65 to 85% w/w of the total weight of the capsule.

According to a preferred embodiment, the core of the capsule comprises at least 20% of polyunsaturated fatty acid.

According to an embodiment of the invention, the seamless capsule comprises in its core at least one omega-3 long chain polyunsaturated acid and/or one omega-6 long chain polyunsaturated acid. According to a preferred embodiment, the core of the capsule includes DHA, EPA or a mixture thereof. The PUFAs entering into the composition of the core of the capsule may be of any origin, and for example issued from vegetable or animal origin. The choice of the source of PUFA will be made in accordance with the final use for which the capsules are produced. For human consumption, it may be preferred to use PUFA, especially DHA, obtained from microalgae or microalgae culture. For animal consumption, PUFA, especially DHA and/or EPA, may be obtained from fish oil or fungal oil.

It is one goal of the invention that the capsule of the invention does not smell or taste the known fish odour and taste of the PUFAs. For this reason, as the fishy odour is essentially due to the oxidized form of the PUFAs, the PUFAs are encapsulated in the capsule of the invention, which shell is barrier to the gaz: the encapsulation results into avoiding the oxidation due to the ambient air of the materials present in the core of the capsule. However, as the capsule of the invention are aimed to be consumed by animals or humans, it is a further problem to avoid for the consumer the fishy taste and odor of the PUFAs at the time of consumption, where the capsule may be broken when eaten.

The solution proposed by the Applicant is to include in the core, together with the PUFAs, at least one flavouring component. By flavouring component in the meaning of this invention, is meant one molecule releasing a fragrance or an aroma, more than one molecule each releasing a fragrance or an aroma, or a combination of molecules, said combination releasing a fragrance or an aroma. According to this invention, the flavouring component is chosen among those able to mask the fishy odour, smell and/or taste of the PUFAs. The flavouring component preferably comprises a sweet note, a brown note, a citrus note, a red fruit note, a meaty note or a mixture thereof. According to an embodiment of the invention, the flavouring component is selected among aromatic or fragrancing molecules as conventionally used in the formulation of flavoring or fragrancing compositions. Mention will in particular be made of aromatic, terpenic and/or sesquiterpenic hydrocarbons, and more particularly essential oils, oleoresin and natural extracts, alcohols, aldehydes, phenols, carboxylic acids in their various forms, aromatic acetals and ethers, nitrogenous heterocycles, ketones, sulfides, disulfides and mercaptans which may be aromatic or nonaromatic. More precisely flavouring component may be selected in the group consisting of vanillin; furaneol®; ethyl maltol; diacetyl and/or methylcyclopentenolone; frambinone; benzaldehyde; lactone; spices; oleoresin such as cinnamon and/or ginger extracts; absolute such as fenugreek and/or oak mousse; citrus essential oils such as lemon and/or orange; fatty acid such as butyric acid; sulphur compounds, such as bisulphite or dimethylsulfur; or a mixture of two or more thereof.

According to the invention, the at least one flavouring component has to be included in the core of the capsule in an amount efficient for masking the fishy odour, smell and taste of the PUFAs present in said the core. Advantageously, the flavouring component(s) represent(s) from 1 to 50%, preferably from 3 to 30% and more preferably 5 to 20% by weight of the total weight of the core. According to an embodiment, the weight ratio of polyunsaturated fatty acid to the flavouring component(s) is of 99:1 to 50:50.

According to an embodiment of the invention, the seamless capsule comprises 0.01 to 200 mg, preferably 0.02 to 50 mg and more preferably 0.03 to 6 mg of omega-3 long chain polyunsaturated acids, preferably of DHA.

According to an embodiment of the invention, the core of the seamless capsule also includes at least one diluent, wherein both the PUFAs and the molecules releasing a fragrance or an arome may be solubilized. According to an embodiment, the diluent comprises an ester such as triacetine or ethylacetate or triethylcitrate, an alcool such as ethylalcohol, a triglycerid such as medium-chain triglycerides, an oil such as sunflower oil, coconut oil, palm oil, lecithin oil, food grade silicone oil, canola oil, flax oil, or a mixture thereof.

According to an embodiment of the invention, the core of the seamless capsule may also contain antioxidant such as tocopherol acetate, rosemary extract, BHA, ascorbyl palmitate and mixture thereof.

According to an embodiment of the invention, the shell of the capsule comprises gelatine, alone or in combination with another gelling agent and plasticizer such as sorbitol, glycerin, mannitol, propylene glycol or any other polyol.

According to another embodiment of the invention, the shell of the capsule comprises gellan gum alone or in combination with another gelling agent, a filler, and a divalent metal sequestering agent.

According to an embodiment, the capsule may be further coated with a film-forming polymer with moisture barrier properties which is at least one hydrophobic agent selected from those suitable for confectionery or pharmaceutical products, preferably selected from the group consisting of waxes, especially carnauba wax, candelilla wax or beeswax, carbowax, shellac (in alcoholic or aqueous solution), cellulose derivatives such as ethyl cellulose, hydroxypropylmethylcellulose, hydroxypropylcellulose and mixtures, polyvinyl alcohol and derivatives, zein, chitosan or modified chitosan or a combination thereof.

The invention also relates to a process for the manufacturing of a capsule comprising:
mixing flavouring component, diluant and PUFA to prepare a core preparation and keeping said preparation in a manner to prevent any moisture contact during the process, (vacuum, nitrogen, argon),
co-extruding an external and hydrophilic liquid phase and an internal and lipophilic liquid phase, in order to form a capsule made of a core comprising the internal and lipophilic phase, and a shell comprising the external and hydrophilic phase,
a centrifugation step to remove excess of oil,
and optionally immersing the resulting capsules into an aqueous solution containing a curing agent
a drying step.
and optionally a coating step The invention also relates to a lipophilic composition suitable for use in combination with a hydrophilic phase comprising gelatine, gellan or a combination of gelatine, gellan and optionally further gelling agent, for the manufacturing of the capsule of the invention having a core and a shell, wherein the core comprises a PUFA and at least one flavouring component.

According to an embodiment of the invention, the lipophilic composition includes at least one PUFA, preferably at least one omega-3 long chain polyunsaturated acid, preferably selected from the group consisting of DHA, EPA, and a mixture thereof; at least one flavouring component; preferably at least one diluent wherein both the at least one PUFA and the at least one flavouring component may be solubilized, said diluent preferably being selected among the group consisting of an ester such as triacetine or ethylacetate or triethylcitrate, an alcool such as ethylalcohol, a triglycerid such as medium-chain triglycerides, an oil such as sunflower oil, coconut oil, palm oil, or a mixture of two or more thereof. According to a preferred embodiment, the diluent includes medium-chain triglycerides, ethanol, triacetine or a mixture of two or more thereof.

According to an embodiment of the invention, the lipophilic composition is a clear limpid homogeneous solution.

The capsules of the invention may enter in the composition of a food fortified, pharmaceutical, nutraceutical, dietetic or nutritional product. This invention thus relates to a pharmaceutical, nutraceutical, dietetic or nutritional product comprising at least one capsule of the invention. According to another embodiment this invention also relates to a pet food product comprising at least one capsule of the invention, in such case a meat flavour will be applied to the capsule. According to an embodiment, the pharmaceutical, nutraceutical, dietetic or nutritional or food product of the invention comprises 0.05 to 100%, preferably 0.08 to 5%, and more preferably 0.1 to 1% by weight of capsules relative to the weight of the commercial product. According to one embodiment of the invention, the pharmaceutical, nutraceutical, dietetic or nutritional or food product of the invention comprises 1 to 50% by weight of capsules relative to the weight of the commercial product.

### EXAMPLE 1

### LEMON Omega3 Capsule

A core solution is prepared with the following formula and is kept at room temperature under nitrogen:
Fish oil rich in PUFA Omegavie from POLARIS 70%
BHA 0.2%
Oleic sunflower oil rich in oleic acid 5%
Lemon flavour from MANE 24.8%

A film solution is prepared with the following formula and is heated for 4 h at 60° C.:
Fish gelatine 275 bloom 19.8%
Gellan gum KELCO F 0.01%
Sorbitol Neosorb from Roquette 2.7%
Water 77.49%

Both solutions are extruded to obtain seamless spherical capsule of a size of 3 mm with a core loading of 85%. Obtained capsules have a total weight of 14 mg and can supply 24% per weight of PUFA. Capsule present a pleasant lemon taste which cover fishy odor.

### EXAMPLE 2

### RASPBERRY PUFA Capsule

A core solution is prepared with the following formula
Lonza DHA-CL DHA-CL® from Lonza 50%
Raspberry flavour from MANE 17.82%
Miglyol 812S from Huls 27.18%
Ethanol 5%

A film solution is prepared with the following formula and is heated for 4 h at 60° C.:
Gelatine Pork type 250 A 19.8%
Sorbitol Neosorb from Roquette 2.7%
Water 77.5%

Both solutions are extruded to obtain perfect seamless spherical capsule of a size of 1 mm with a core loading of 68%. Total weight of each capsule is 0.7 mg and can supply 34% PUFA per capsule. Capsule present a pleasant raspberry smell and is incorporated into ceral bar product at a dosage of 0.3% supplying 50 mg PUFA for 50 g serving size.

1. A seamless capsule comprising a core and a shell, wherein the core comprises at least one polyunsaturated fatty acid, and at least one flavouring component.

2. The seamless capsule according to claim 1, wherein the core further includes at least one diluent wherein both the at least one fatty acid and the at least one flavouring component may be solubilized.

3. The seamless capsule according to claim 1, wherein the diameter or said capsule is from 0.8 to 8 mm, preferably from 1 to 3 mm.

4. The seamless capsule according to claim 1, wherein the dry weight of the capsule is from 0.25 to 260 mg, preferably from 0.5 to 100 mg, more preferably from 0.7 to 15 mg.

5. The seamless capsule according to claim 1, wherein the core represents 50 to 92% by weight of the total weight of the capsule.

6. The seamless capsule according to claim 1, wherein the core comprises at least 20% of polyunsaturated fatty acid.

7. The seamless capsule according to claim 1, wherein said at least one fatty acid comprises at least one omega-3 long chain polyunsaturated acid and/or one omega-6 long chain polyunsaturated acid.

8. The seamless capsule according to claim 1, wherein said at least one fatty acid comprises DHA, EPA or a mixture thereof.

9. The seamless capsule according to claim 8, wherein the DHA and/or the EPA is of vegetable or animal origin, preferably from microalgae or microalgae culture, or from fish oil or fungal oil.

10. The seamless capsule according to claim 1, wherein the at least one flavouring component is chosen among those masking the odour, smell and/or taste of said PUFA.

11. The seamless capsule according to claim 10, wherein the at least one flavouring component comprises a sweet note, brown note; citrus note, a red fruit note, a meaty note or a mixture thereof.

12. The seamless capsule according to claim 10, wherein the flavouring component is selected among the group consisting of vanillin; furaneol; ethyl maltol; ketone such as diacetyl and/or methylcyclopentenolone; frambinone; benzaldehyde; lactone type; spices oleoresin such as cinnamon and/or ginger extracts; absolute such as fenugreek and/or oak mousse; citrus essential oils such as lemon and/or orange; fatty acid such as butyric acid; sulphur compounds, such as bisulphite or dimethylsulfur; or a mixture of two or more thereof.

13. The seamless capsule according to claim 2, wherein the at least one diluent comprises an ester such as triacetine or ethylacetate or triethylcitrate; an alcool such as ethylalcohol; a triglycerid such as medium-chain triglycerides; an oil such as sunflower oil, coconut oil, palm oil; lecithin oil, food grade silicone oil, canola oil, flax oil or a mixture thereof.

14. The seamless capsule according to claim 1, wherein the at least one flavouring component represents from 1 to 50%, preferably from 3 to 30% and more preferably from 5 to 20% by weight of the total weight of the core.

15. The seamless capsule according to claim 1, wherein the weight ratio of polyunsaturated fatty acid to the flavouring component is of 99:1 to 50:50.

16. The seamless capsule according to claim 7, comprising 0.01 to 200 mg, preferably 0.02 to 50 mg, more preferably 0.03 to 5 mg of omega-3 long chain polyunsaturated acids, preferably of DHA.

17. The seamless capsule according to claim 1, wherein the core of the seamless capsule also contain antioxidant such as tocopherol acetate, rosemary extract, BHA, ascorbyl palmitate and mixture thereof.

18. The capsule according to claim 1, wherein the shell comprises gelatin, alone or in combination with another gelling agent and plasticizer such as sorbitol, glycerin, mannitol, propylene glycol or any other polyol.

19. The capsule according to claim 1, wherein the shell comprises gellan gum alone or in combination with another gelling agent, a filler, and a divalent metal sequestering agent.

20. The capsule according to claim 1, wherein said capsule is further coated with a film-forming polymer selected from the group consisting of cellulose derivatives, waxes, polyvinylalcohol.

21. Process for the manufacturing of a capsule comprising
mixing flavouring component, diluant and PUFA to prepare a core preparation and keeping said preparation in a manner to prevent any moisture contact during the process,
co-extruding an external and hydrophilic liquid phase and an internal and lipophilic liquid phase, in order to form a capsule made of a core comprising the internal and lipophilic phase, and a shell comprising the external and hydrophilic phase,
a centrifugation step to remove excess of oil,
and optionally immersing the resulting capsules into an aqueous solution
and a drying step.

22. Lipophilic composition comprising:
at least one polyunsaturated fatty acid,
at least one flavouring component
optionally, at least one diluent wherein both the at least one fatty acid and the at least one flavouring component may be solubilized.

23. The lipophilic composition according to claim 22, wherein said at least one PUFA comprises at least one omega-3 long chain polyunsaturated acid, preferably selected from the group consisting of DHA, EPA, and a mixture thereof.

24. The lipophilic composition according to claim 22, wherein the DHA and/or the EPA is of vegetable or animal origin, from microalgae or microalgae culture or from fish oil or fungal oil.

25. The lipophilic composition according to claim 22 wherein said lipophilic composition is a clear limpid homogeneous solution.

26. The lipophilic composition according to claim 22, wherein the at least one diluent comprises an ester, an alcool, a triglyceride, an oil or a mixture thereof.

27. The lipophilic composition according to claim 22, wherein the at least one diluent comprises medium-chain triglycerides, ethanol, triacetine or a mixture thereof.

28. Use of a lipophilic composition according to claim 22, in combination with an hydrophilic phase comprising gelatine, gellan, or a combination thereof, for the manufacturing of capsule having a core and a shell, wherein the core comprises DHA and a flavouring component.

29. Food fortified, pharmaceutical, nutraceutical, dietetic, nutritional or food compositions comprising at least one capsule according to claim 1.

30. Food fortified, pharmaceutical, nutraceutical, dietetic, nutritional or food compositions according to claim 29, comprising 0.05 to 100%, preferably 0.08 to 5%, and more preferably 0.1 to 1% by weight of capsules relative to the weight of the commercial product.

31. Food product according to claim 29, wherein said food product is a pet food product.
